(19) United States
(12) Patent Application Publication
(10) Pub. No.: US 2025/0009709 A1
(43) Pub. Date: Jan. 9, 2025

(54) PRENYLATED CHALCONE AND FLAVONOID COMPOSITIONS FOR USE IN TREATING CANCER

(71) Applicant: INNOX CORP., Mahwah, NJ (US)

(72) Inventors: Devrim ARAN, Mahwah, NJ (US); Robert NIZIOL, Mahwah, NJ (US); Mariusz GUJSKI, Mahwah, NJ (US)

(73) Assignee: INNOX CORP., Mahwah, NJ (US)

(21) Appl. No.: 18/577,633

(22) PCT Filed: Jul. 8, 2022
(86) PCT No.: PCT/US2022/036485

§ 371 Date: Jan. 8, 2024

Related U.S. Application Data
(60) Provisional application No. 63/220,054, filed on Jul. 9, 2021.

(51) Int. Cl. A61K 31/353; A61K 9/16; A61K 31/00; A61K 31/12; A61K 31/506; A61P 35/02

(52) U.S. Cl. CPC A61K 31/353; A61K 9/1617; A61K 9/1635; A61K 9/1652; A61K 31/12; A61K 31/506; A61K 31/658; A61K 36/3486; A61P 35/02; A61K 2236/333; A61K 2236/39; A61K 2236/51

(57) ABSTRACT

Provided are compositions comprising xanthohumol and isoxanthohumol and their use in treatment of cancer, for example, tyrosine kinase inhibitor resistant cancers.

### BACKGROUND OF THE DISCLOSURE

At present, treatment modes for cancer mainly comprise surgery, chemotherapy, radiotherapy, molecularly targeted therapy, gene therapy, and immunotherapy traditionally targeting a single biological target. However, the curative effects of these treatments have been limited thus far by specific characteristics of tumors.

Drug resistance inevitably limits the efficacy of all targeted therapies. For tumor cells, drug resistance to tyrosine kinase inhibitors (TKIs) is a major obstacle in both solid tumor and leukemia/lymphoma. Tumor cells can be TKI-sensitive or TKI-refractory, exhibit intrinsic or acquired resistance, and accumulate alterations within or outside the target to promote their survival.

Xanthohumol is a prenylated chalcone derived from hops, more specifically the female hop plant. Xanthohumol exhibits a broad range of bioactivities, including modulating key transcription factors but mostly renowned for its anti-oxidant activity. It is thus considered useful in the treatment of diseases associated with oxidative stress such as for example cancer, diabetes and dyslipidemia

Current xanthohumol-containing products have at least two disadvantages. First, the bioavailability of xanthohumol in these products is very poor due to its low water-solubility. Second, xanthohumol products often contain a substantial amount of isoxanthohumol, which results from the breakdown of xanthohumol during preparation (e.g. in heating steps) or storage of xanthohumol-containing products. Thus, there is a need to develop new formulations that contain high amounts of bioavailable xanthohumol for treatment of cancer, such as those that are drug resistant.

### SUMMARY OF THE DISCLOSURE

The present disclosure is directed to a pharmaceutical composition comprising a therapeutically-effective amount of xanthohumol and isoxanthohumol and at least one pharmaceutically acceptable carrier. In one aspect, the composition further comprising 6-prenylnaringenin and 8-prenylnaringenin. In another aspect, the the xanthohumol is present in an amount between at least 50-99% w/w, the isoxanthohumol is present in an amount between 1-15% w/w, 6-prenylnaringenin is present in an amount between 0.0-5%, and 8-prenylnaringenin is present in an amount between 0.0-5%.

In one aspect, at least one of the xanthohumol, isoxanthohumol, 6-prenylnaringenin, or 8-prenylnaringenin is extracted from a botanical. In another aspect, the botanical is hops, hops spent, or any hops-containing product. In another aspect, at least one of xanthohumol, isoxanthohumol, 6-prenylnaringenin, 8-prenylnaringenin is chemically synthesized.

In one aspect, the composition of the disclosure comprises a pharmaceutically acceptable carrier which is at least one of a binder, disintegrant, surfactant, or lubricant. In another aspect, the binder is one or more of starch 1500, polyvinylpyrolidone, and microcrystalline cellulose. In another aspect, the binder is present in an amount of between 10-30% w/w. In another aspect, the disintegrant is croscarmellose sodium. In another aspect, the disintegrant is present in an amount of between 1-5% w/w. In another aspect, the surfactant is sodium dodecyl sulfate. In another aspect, the surfactant is present in an amount of between 1-5% w/w. In another aspect, the lubricant is magnesium stearate. In another aspect, the lubricant is present in an amount of between 0.5-3% w/w.

The present disclosure is also directed to a method of preparing a composition comprising xanthohumo and isoxanthohumol from hops comprising: suspending the hops plant material in n-heptane to remove non-polar impurities; evaporating the heptane and filtering the residue; extracting the residue with ethyl acetate and collecting the xanthohumol-containing fraction in a rotary evaporator; extracting the xanthohumol with an organic acid solution, containing 0.05M ZnCl₂, 5% NaHCO₃ and brine; drying the organic layer with anhydrous Na₂SO₄; precipitating the xanthohumol from the mixture containing ethyl acetate; and drying the precipitated xanthohumol and isoxanthohumol. The present disclosure is also directed to a composition comprising the xanthohumol and isoxanthohumol obtained by the process.

The present disclosure is also directed to a method of agonizing farnesoid X receptor activity in a subject in need thereof, comprising administering a therapeutically effective amount of a composition described herein to the subject. In one aspect, agonizing farnesoid X receptor activity results in treatment of cancer. In another aspect, the cancer is tyrosine kinase inhibitor resistant.

The present disclosure is also directed to a method of inhibiting NFκB activity in a subject in need thereof, comprising administering a therapeutically effective amount of a composition described herein to the subject. In one aspect, inhibiting NFκB activity results in treatment of cancer. In another aspect, the cancer is tyrosine kinase inhibitor resistant.

The present disclosure is also directed to a method of modulating expression and/or activation of nuclear factor erythroid 2-related factor 2 (NRF2) in a subject in need thereof, comprising administering a therapeutically effective amount of a composition described herein to the subject. In one aspect, modulating expression and/or activation of NRF2) results in treatment of cancer. In another aspect, the cancer is tyrosine kinase inhibitor resistant.

The present disclosure is also directed to a method of inducing apoptosis in a cell, comprising contacting the cell with an effective amount of a composition described herein. In one aspect, the cell is a leukemic cell. In another aspect, the cell contains the bcr-abl gene mutation. In another aspect, the cell is in a patient having chronic myelogenous leukemia (CML), acute lymphoblastic leukemia (ALL), or acute myelogenous leukemia (AML).

The present disclosure is also directed to a method of treating chronic

myelogenous leukemia (CML) in a subject, comprising administering a therapeutically effective amount of a composition described herein to the subject. In one aspect, the CML is resistant to therapy with a tyrosine kinase inhibitor (TKI). In another aspect, the TKI is imatinib, dasatinib, or ponatinib.

The present disclosure is also directed to a method of decreasing the incidence of secondary tyrosine kinase inhibitor (TKI) resistance in a subject comprising administering a therapeutically effective amount of a composition described herein to the subject.

The present disclosure is also directed to a method of treating BCR-ABL-independent resistant cancer in a subject comprising administering a therapeutically effective amount of a composition described herein to the subject. In one aspect, the BCR-ABL-independent resistance is caused by inhibition of CRKL and STAT5phosphorylation, or sustained phosphorylation of the translation regulator ribosomal protein S6 (RPS6) indicated activation of mTOR complex 1 (mTORC1).

In one aspect of the disclosure, the methods further comprise administering a TKI to the subject. In another aspect, the TKI is administered after the composition. In another aspect, the TKI is imatinib, dasatinib, ponatinib, or nilotinib.

In one aspect of the disclosure, the methods further comprise administering a cannabinoid to the subject. In another aspect, the cannabinoid is administered after the composition.

### BRIEF DESCRIPTION OF THE DRAWINGS

FIG. 1 shows a block diagram of the process of purifying a xanthohumol-containing drug substance from lemyelins.

FIGS. 2A-2C show particle size distribution for XN-54 (FIG. 2A), XN-63-10 (FIG. 2B), and XN-87 (FIG. 2C).

FIGS. 3A-3B show the differential scanning calorimetry (DSC) for batch XN-54 (FIG. 3A) and for DS batch XN-87 (FIG. 3B).

FIGS. 4A-4B show the thermogravimetric analyses for batch XN-54 (FIG. 4A) and for batch XN-87 (FIG. 4B).

FIGS. 5A-5B show the Fourier Transformed Infrared analyses (FTIR) of batch XN-54 (FIG. 5A) and XN-87 (FIG. 5B).

FIGS. 6A-6B show the X-ray powder diffractogram for batch XN-54 (FIG. 6A) and for batch XN-87 (FIG. 6B).

FIGS. 7A-7B show pCrkl (FIG. 7A) and BCR-ABL (FIG. 7B) levels in K562-DR1000 nM cells.

FIGS. 8A-8B show the effect of increasing concentrations of XN-54 or dasatinib on cell viability at 48 hours (FIG. 8A) and 72 hours (FIG. 8B) in K562-IS,-IR, and -DR cells.

FIGS. 9A-9B show XN-54 induced apoptosis in K562-IS,-IR, and -DR cells as measured using early apoptosis markers at 48 hours (FIG. 9A) and 72 hours (FIG. 9B).

FIGS. 10A-10B show XN-54 induced apoptosis in K562-IS,-IR, and -DR cells as measured using late apoptosis markers at 48 hours (FIG. 10A) and 72 hours (FIG. 10B).

FIGS. 11A-11B show the effect of a combination of XN-54 and ponatinib on cell viability in K562-IR cells based on either log 10(M) XN-54 (FIG. 11A) or ponatinib (FIG. 11B).

FIGS. 12A-12B show the effect of a combination of XN-54 and nilotinib on cell viability in K562-IS cells based on either log 10(M) nilotinib (FIG. 12A) or XN-54 (FIG. 12B).

FIGS. 13A-13C show the effect of a combination of XN-54 (Compound 1) and cannabidiol (CBD) (Compound 2) on cell viability in K562-DR (FIG. 13A), K562-IS (FIG. 13B), and K562-IR cells (FIG. 13C).

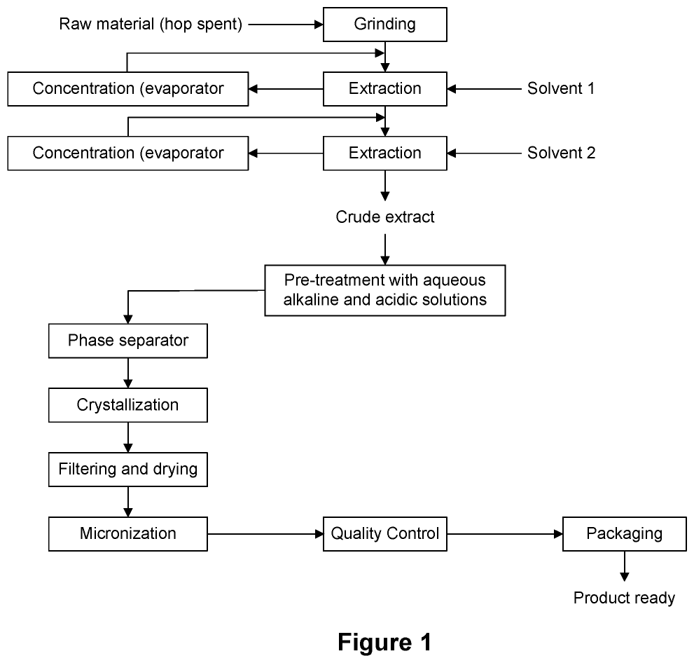

### DETAILED DESCRIPTION

The present disclosure is directed to compositions comprising xanthohumol, isoxanthohumol, 6-prenylnaringenin, 8-prenylnaringenin and their use in treatment of cancer. In some aspects, the compositions are useful for treating leukemia and solid tumors. In other aspects, the compositions are useful in treating drug resistant cancers.

### General Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

### Pharmaceutical Compositions

In the context of the present disclosure, the term “xanthohumol” is to be understood as meaning a prenylated chalconoid obtainable from botanicals, such as hop, hops spent or hops products, and is represented by the following formula:

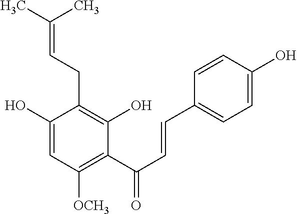 [SMILES: COc1cc(O)c(CC=C(C)C)c(O)c1C(=O)/C=C/c1ccc(O)cc1]

Xanthohumol as used in the present disclosure may be a commercially available pure form of the molecule, or alternatively be an (enriched) extract obtained from a suitable source such as a botanical.

In the context of the present disclosure the term “isoxanthohumol” is to be understood as the corresponding prenylated flavanone of xanthohumol, and is represented by the following formula:

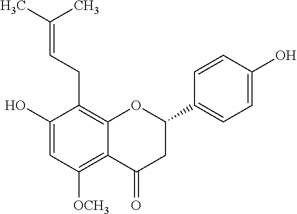 [SMILES: COc1cc(O)c(CC=C(C)C)c2c1C(=O)C[C@@H](c1ccc(O)cc1)O2]

6-prenylnaringenin is a trihydroxyflavanone having a structure of naringenin prenylated at C-6. It is a trihydroxyflavanone, a member of 4′-hydroxyflavanones and a (2S)-flavan-4-one. It derives from a(S)-naringenin. It is represented by the following formula:

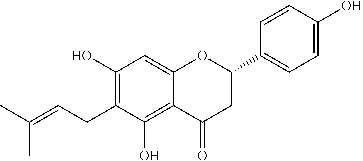 [SMILES: CC(C)=CCc1c(O)cc2c(c1O)C(=O)C[C@@H](c1ccc(O)cc1)O2]

8-prenylnaringenin or sophoraflavanone B is a trihydroxyflavanone that is(S)-naringenin having a prenyl group at position 8. It has a role as a platelet aggregation inhibitor and a plant metabolite. It is a trihydroxyflavanone, a member of 4′-hydroxyflavanones and a (2S)-flavan-4-one. It derives from a (S)-naringenin. It is a conjugate acid of a sophoraflavanone B(1-). It is represented by the following formula:

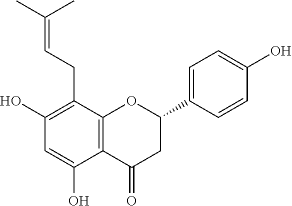 [SMILES: CC(C)=CCc1c(O)cc(O)c2c1O[C@H](c1ccc(O)cc1)CC2=O]

As used herein, the terms xanthohumol, isoxanthohumol, 6-prenylnaringenin, and 8-prenylnaringenin, and the like, include derivatives such as hydroxylated and sulfates prenylated flavonoids, or can be polymorphs, or in any solid or liquid physical form. For example, the compounds can be in a crystalline form, in amorphous form, and have any particle size. The particles may be micronized, or may be agglomerated, particulate granules, powders, oils, oily suspensions or any other form of solid or liquid physical form.

The processes for extracting and purifying xanthohumol, isoxanthohumol, 6-prenylnaringenin and 8-prenylnaringenin from plant material described in the literature have a number of disadvantages. For example, large amounts of organic solvents, and in particular highly toxic solvents (dichloromethane, hexane, chloroform) are needed for efficient extraction. Current methods also employ large amounts of expensive materials, e.g. silica gel. The methods cause irreversible decomposition of xanthohumol to by-products (mainly isomeric isoxanthohumol) and to polymeric oxidative degradation products (extraction with aqueous solution of strong bases—e.g. NaOH, KOH). Countercurrent chromatography techniques have low efficiency and scalability of the process. Most importantly, it is nearly impossible to obtain xanthohumol with high pharmaceutical purity (min. 95% by weight). However, the present disclosure provides a process for preparing xanthohumol with an HPLC purity >95%.

The known methods of obtaining xanthohumol and others by chemical synthesis are inefficient and not very scalable. Moreover, the final stage of the littering does not allow for the economically viable preparation of large amounts of xanthohumol with high pharmaceutical purity.

In some aspects, the disclosure provides a process for preparing xanthohumol from botanicals. In some aspects, the botanical is hops. Hop or hops (Humulus lupulus) is a climbing vine belonging to the genus Humulus in the family Cannabaceae, order Urticacales. Older taxonomists included the genera Humulus in the mulberry family (Moraceae).

Hops is a dioecious perennial plant native to the Northern Hemisphere. It is found in shrubbery and at the edge of forests with access to sufficient water, and it reaches a height of up to 7-8 m (23-26 feet). Many female flowers form an inflorescence, called strobiles, which consist of membranous stipules and bracts that are attached to a zigzag, hairy axis. The bracts and stipules of the hop contain polyphenols; the odor and taste of the drug is due mainly to the very complex secretion contained in the lupulin glands.

After harvesting, the inflorescences are dried immediately to a water content of about 10% for stability reasons. Also, depending on the environmental conditions, hops is kept under constant refrigeration during some or all steps from harvest to final product. The bitter principles are known to break down rapidly during storage and, unless refrigerated, their concentration decreases by 50 to 70% in only 6 months.

Twenty-five percent of the harvested hop strobiles are extracted with ethanol or supercritical carbon dioxide to obtain as many alpha acids as possible. Since ethanol and carbon dioxide are naturally occurring during the brewing process, the use of these solvents is of no concern.

From the immense biomass production, the inflorescences (strobiles) are the only part of the hops plant that is used. Except for some use of young shoots, eaten in salads, there has been no human use for the stems, leaves, rhizomes, and roots. The above-ground (aerial) parts are composted and used for fertilization of the fields. However, using the current process, xanthohumol and other molecules are purified from hops, hops waste generated from other processes, such as beer production (hops spent), or any hops-containing product in biologically active and safe ratios.

In certain aspects, the compositions of the disclosure are formulated for administration in a therapeutically effective amount. The terms “effective amount,” “pharmaceutically effective amount” and “therapeutically effective amount” refer to an amount that may be effective to elicit the desired biological or medical response. including the amount of a compound that, when administered to a subject for treating a disease, is sufficient to affect such treatment for the disease. The effective amount will vary depending on the compound, the disease and its severity and the age, weight, etc., of the subject to be treated. The effective amount can include a range of amounts. Further, an effective amount includes amounts of an agent which are effective when combined with other agents.

In some aspects, the effective amount of the composition is combined with one or more pharmaceutically acceptable vehicles. The term “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable vehicles (e.g., carriers, adjuvants, and/or other excipients) have preferably met the required standards of toxicological and manufacturing testing and/or are included on the Inactive Ingredient Guide prepared by the U.S. Food and Drug administration.

The term “carrier” or “pharmaceutically acceptable carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin. Examples of carriers may include, but are not limited to, aluminum monostearate, aluminum stearate, carboxymethylcellulose, carboxymethylcellulose sodium, croscarmellose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, lactose monohydrate, polysaccharides, cellulose, cellulose ethers, hydroxypropyl cellulose, microcrystalline cellulose, sugar alcohols, xylitol, sorbitol, maltitol, compressible sugars, calcium or sodium carbonate, dicalcium phosphate, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “lubricant” refers to a substance added to a powder blend to prevent the compacted powder mass from sticking to the equipment during the tableting or encapsulation process. A lubricant can aid the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C₈/C₁₀ fatty acid.

The term “film coating” refers to a thin, uniform, film on the surface of a substrate (e.g., tablet). Film coatings are particularly useful for protecting the active ingredient(s) from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

### Methods of Use

Prenylated chalcones and flavonoids have gained increasing attention not only in nutrition but also in disease prevention because of their biological and molecular activities in humans. In one aspect of the disclosure, the compositions described herein are useful in treating/preventing a variety of diseases and conditions including cancer, particularly leukemias such as TKI-resistant chronic myelogenous leukemia (CML).

CML is a myeloproliferative neoplasia associated with a molecular alteration, the fusion gene BCR-ABL1, that encodes the tyrosine kinase oncoprotein BCR-ABL1. This led to the development of TKIs, with Imatinib being the first TKI approved for therapeutic use. Although the vast majority of CML patients respond to Imatinib, resistance to this targeted therapy contributes to therapeutic failure and relapse.

Resistance to targeted therapy is a complex and multifactorial process that culminates in the selection of a cancer clone with the ability to evade treatment. In CML, TKI resistance mechanisms are usually subdivided into BCR-ABL1 dependent and independent mechanisms (Morozova E. V. et al. Biomark. Insights. 2015;10:43-47). However, in therapeutic guidelines, only BCR-ABL1 related mechanisms are taken into consideration for dose adjustments or TKI switch (Baccarani M. et al. 2013. Blood. 2013;122:872-884.) The persistence of leukemic stem cells (LSCs) and LSC-like phenotype based on BCR-ABL1 protein suppression have also been reported as a main TKI resistance mechanisms (Baykal-Köse S. et al. PLoS ONE. 2020;15:e0229104.)

While not being tied to a mechanism, in some aspects, the compositions of the disclosure are useful in agonizing the Farnesoid X receptor. The Farnesoid X receptors (FXR) belong to a family of receptors known as the nuclear hormone receptors. FXR is a bile acid-activated nuclear receptor that is widely implicated in tumorigenesis.

FXR activation down-regulates the breast cancer target genes; local estrogen producer aromatase and the transporters MDR3, MRP-1, solute carrier transporter 7A5(SLC7A5); and inhibits cell proliferation (Bishop-Bailey, D., et al. Cancer Res (2006) 66 (20): 10120-10126). It also induces the expression of the known FXR target genes SHP, IBABP, and MRP2. It has been shown to modulate several cell-signaling pathways such as EGFR/ERK, NF-κB, p38/MAPK, PI3K/AKT, Wnt/β-catenin, and JAK/STAT along with their targets such as caspases, MMPs, cyclins; tumor suppressor proteins like p53, C/EBPβ, and p-Rb; various cytokines; EMT markers; and others.

In some aspects, the compositions of the disclosure are useful in inhibiting NFκB activity in a cell. Nuclear factor kappa B (NF-κB) is an ancient protein transcription factor (Salminen, A., Huuskonen, et al. (2008). Ageing Res. Rev. 7, 83-105) and considered a regulator of innate immunity (Baltimore, D. (2009). Cold Spring Harb. Perspect. Biol. 1:a000026.). NFκB is also an important signaling pathway involved in pathogenesis and treatment of cancers (Xia, L., et al. Onco Targets Ther. (2018) 11:2063-2073).

In some aspects, the compositions of the disclosure are useful in modulating expression/activity of nuclear factor-erythroid 2 (NF-E2) related factor 2 (Nrf2). The Nrf2/Keap1 pathway is an important signaling cascade responsible for the resistance of oxidative damage induced by exogenous chemicals. It maintains the redox homeostasis, exerts anti-inflammation and anticancer activity by regulating its multiple downstream cytoprotective genes, thereby plays a vital role in cell survival. Interestingly, in recent years, accumulating evidence suggests that Nrf2 has a contradictory role in cancers. Aberrant activation of Nrf2 is associated with poor prognosis. The constitutive activation of Nrf2 in various cancers induces pro-survival genes and promotes cancer cell proliferation by metabolic reprogramming, repression of cancer cell apoptosis, and enhancement of self-renewal capacity of cancer stem cells. More importantly, Nrf2 is proved to contribute to the chemoresistance and radioresistance of cancer cells as well as inflammation-induced carcinogenesis.

In some aspects, the compositions of the disclosure are administered in combination with other therapeutic agents. In one aspect, the compositions of the disclosure are administered in combination with one or more tyrosine kinase inhibitor. Tyrosine kinase inhibitors (TKI) are a group of pharmacologic agents that disrupt the signal transduction pathways of protein kinases by several modes of inhibition. Mutations, dysregulation, and overexpression of protein kinases are involved in a multitude of disease processes. Around 1 in every 40 human genes codes for a protein kinase, and nearly half of those genes map to either disease loci or cancer amplicons. Interest in protein kinase inhibitors began with the FDA approval of the tyrosine kinase inhibitor (TKI) imatinib in 2001. Imatinib is an oral chemotherapy medication designed to target the BCR-Abl hybrid protein, a tyrosine kinase signaling protein produced in patients with Philadelphia-chromosome-positive chronic myelogenous leukemia.

As a whole, tyrosine kinases phosphorylate specific amino acids on substrate enzymes, which subsequently alter signal transduction leading to downstream changes in cellular biology. The downstream signal transduction set off by TKs can modify cell growth, migration, differentiation, apoptosis, and death. Constitutive activation or inhibition, either by mutations or other means, can lead to dysregulated signal cascades, potentially resulting in malignancy and other pathologies. Therefore, blocking these initial signals via TKIs can prevent the aberrant action of the mutated or dysfunctional TKs.

Kinase inhibitors are either irreversible or reversible. The irreversible kinase inhibitors tend to covalently bind and block the ATP site resulting in irreversible inhibition. The reversible kinase inhibitors can further subdivide into four major subtypes based on the confirmation of the binding pocket as well as the DFG motif.

In some aspects, the compositions of the disclosure are administered in combination with imatinib, dasatinib, or ponatinib.

The inevitable barrier that limits the effectiveness of TKI therapy is the issue of resistance—today's pervasive challenge for long-term disease control. Cancer is at its core a microcosm of evolution. Its survival is driven by genetic diversity and longitudinal accumulation of mutations, influenced by the selective pressures of TKI therapy. These rudimentary yet intricate principles underlie the refractory nature of TKI resistance, which is traditionally categorized as primary (intrinsic) or secondary (acquired). In primary resistance, patients lack any treatment response to targeted therapy. In secondary resistance, patients initially achieve some clinical benefit, followed by disease progression. With the discovery of each oncogenic driver and targeted inhibitor, a growing number and diversity of resistance mechanisms are being defined.

In one aspect of the disclosure, the compositions described herein are administered, alone or in combination with a TKI, to a subject having primary or secondary TKI resistance. In one aspect, the composition of the disclosure is administered as a first-line therapy to avoid development of secondary TKI resistance. In some aspects, the compositions of the disclosure are administered in combination with one or more TKIs, either simultaneously, or in any order.

In some aspects, the compositions of the disclosure are administered in combination with cannabinoids, either simultaneously, or in any order. In one aspect, the cannabinoid is a phytocannabinoid, an endocannabinoid, or a synthetic cannabinoid. In one aspect, the cannabinoids are cannabidiol (CBD), tetrahydro-cannabinol (THC), or cannabigerol (CBG) or other cannabinoids.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

### EXAMPLES

### Purification of Biologically Active Drug Substance

### Input Material

Green-gray dust and granules (production waste, “leaps”) with a xanthohumol content of 0.3-1.0 wt. %. The raw material contains a large amount of non-polar and polar impurities that are difficult to remove in classical extraction processes (water extraction/water-immiscible organic solvent).

### Extraction Procedure

The plant material was suspended in n-heptane and subjected to a continuous extraction process at 20-30° C. in order to remove non-polar impurities. The extraction temperature was increased to 50-60° C. (some components of the plant raw material melt at this temperature) and the process is repeated. The heptane was evaporated and the yellow residue is filtered through a cotton filter with a press. The dry residue was transferred to the extraction reactor, ethyl acetate was added and the process was repeated analogously to the n-heptane extraction, except that the semi-finished product was now collected in the flask on the rotary evaporator. At the end of the process, the xanthohumol content in the yellowish precipitate was about 10-12% by weight.

The precipitate was dissolved in the appropriate amount of organic solvent and liquid alkane mix to obtain a concentration of xanthohumol ˜0.05M. The resulting dark solution was subjected to an extraction process against 0.2M organic acid solution, 0.05M ZnCl₂, 5% NaHCO₃ and brine. After drying the organic layer with anhydrous Na₂SO₄ and evaporating the organic solvent, a solid mixture with a xanthohumol content of 20-25% was obtained. The mixture was then dissolved in enough ethyl acetate to obtain a concentration xanthohumol 0.05-0.1M, then the solution was subjected to filtration and base-acid extraction in flow using a liquid-liquid separator. Briefly, the solution of xanthohumol in ethyl acetate was extracted against 0.05-0.1M NaOH solution, the organic layer was discarded, xanthohumol in the water layer (as sodium salt) was added to 0.05M-0.1M organic acid solution, which causes precipitation of Xn which was extracted into ethyl acetate and dried with anhydrous Na₂SO₄. Ethyl acetate and brine were added to the residue. After filtering off the inorganic salt, most of the ethyl acetate was evaporated off and the calculated amount of n-heptane was added to initiate precipitation of xanthohumol from the solvent mixture. The distillation process was continued until all xanthohumol precipitated. Alcohol was added (to remove traces of ethyl acetate) and the evaporation process continued. The precipitate was filtered off, washed with n-heptane and dried in vacuo to give the final product as a light yellow powder (HPLC purity>95.0%).

### Drug Substance Characterization

Three batches of drug substance (XN-54, XN-63-10, and XN-87) containing xanthohumol, isoxanthohumol, 6-prenylnaringenin, 8-prenylnaringenin were prepared using the above process and analyzed.

### Particle Size Distribution (PSD)

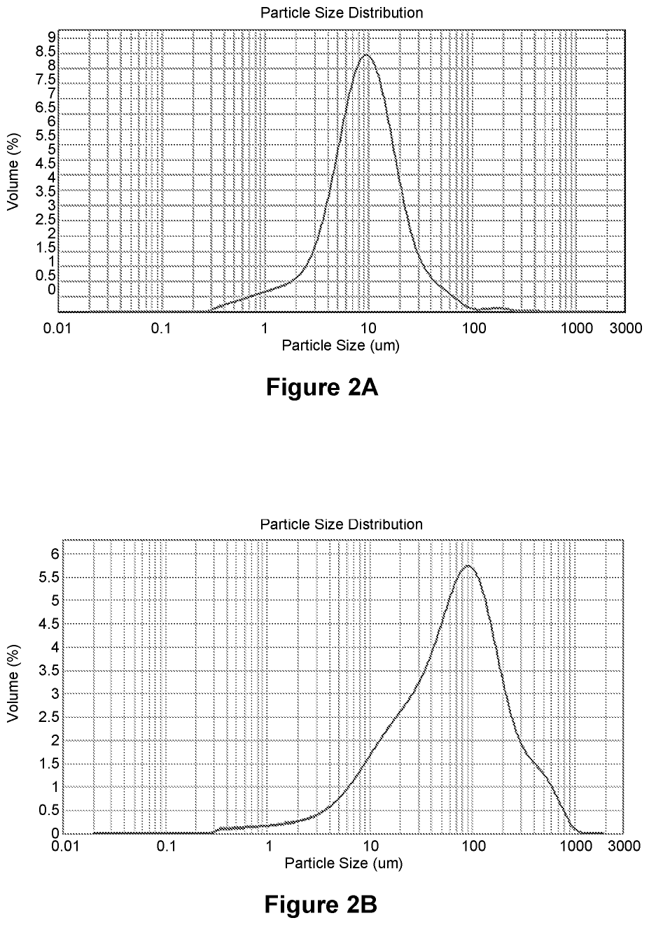

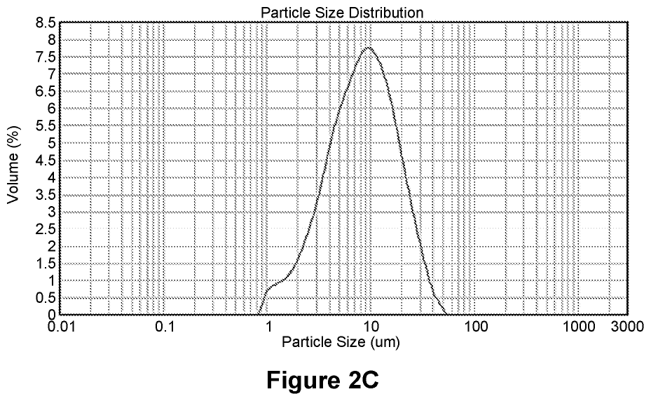

PSD tests were performed by laser diffraction using a Mastersizer 2000 by Malvern analyzer. Table 1 presents average values (in μm) of three tested batches: XN-54 (sample was grinded in a mortar before analysis), XN-63-10 and XN-87. Table 2 summarizes results of batch XN-63 and its micronized derivatives. Particle size distribution for XN-54 (FIG. 2A), XN-63-10 (FIG. 2B), and XN-87 (FIG. 2C) are shown.

TABLE 1
PSD results of the drug substance,
batches: XN-54, XN-63-10 and XN-87

| | XN-54 (grinded) | XN-63-10 (as is) | XN-87 (as is) |
|---|---|---|---|
| d (0.1) | 2.659 | 10.453 | 2.918 |
| d (0.5) | 8.602 | 69.969 | 8.557 |
| d (0.9) | 23.287 | 274.505 | 21.353 |
| D [4.3] | 12.902 | 114.858 | 10.626 |
| D [3.2] | 4.924 | 18.194 | 5.949 |

TABLE 2
PSD results of the drug substance, batch XN-63-10 and its micronized derivatives

| | XN-63-10 (as is) | XN-63-1 | XN-63-4 | XN-63-5 | XN-63-6 | XN-63-7 |
|---|---|---|---|---|---|---|
| d (0.1) | 10.453 | 4.051 | 2.688 | 1.802 | 1.804 | 1.714 |
| d (0.5) | 69.969 | 29.744 | 10.866 | 8.555 | 5.642 | 6.908 |
| d (0.9) | 274.505 | 311.222 | 28.647 | 25.149 | 18.841 | 19.377 |
| D [4.3] | 114.858 | 98.766 | 37.354 | 16.526 | 23.228 | 17.011 |
| D [3.2] | 18.194 | 7.852 | 4.996 | 3.934 | 3.867 | 3.572 |

### Differential Scanning Calorimetry (DSC)

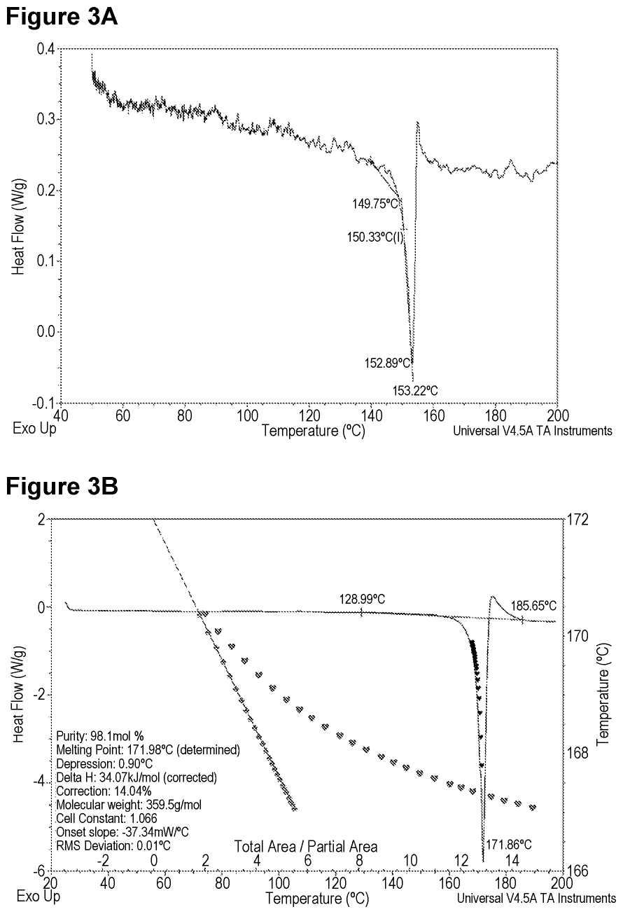

Differential Scanning calorimetry analyses were performed on the TA Instruments Differential Scanning calorimetry (DSC), type DSC Q20 V24.10 Build 122.DSC analysis of two DS batches showed two different melting point results. The melting point for DS batch XN-54 was 153.22° C. (FIG. 3A) and for DS batch XN-87 was 171.86° C. (FIG. 3B).

### Thermogravimetric Analysis (TGA)

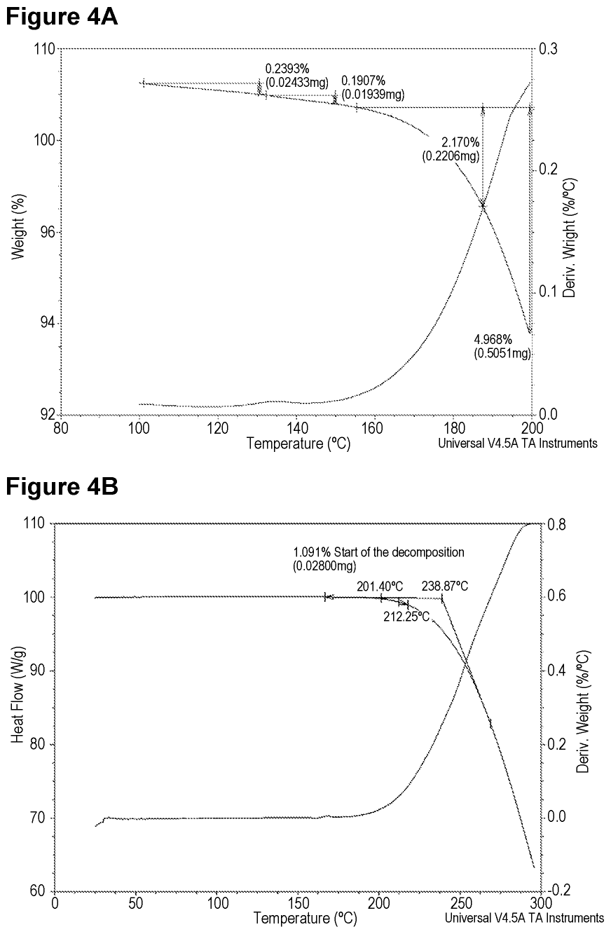

Thermogravimetric analyses were performed on the TA Instruments Thermogravimetric Analyzer (TGA), type Q50 V20.13 Build 39. The TGA of drug substance, batch XN-54 is shown in FIG. 4A and for batch XN-87 in FIG. 4B.

### Fourier Transform Infrared Analysis (FTIR)

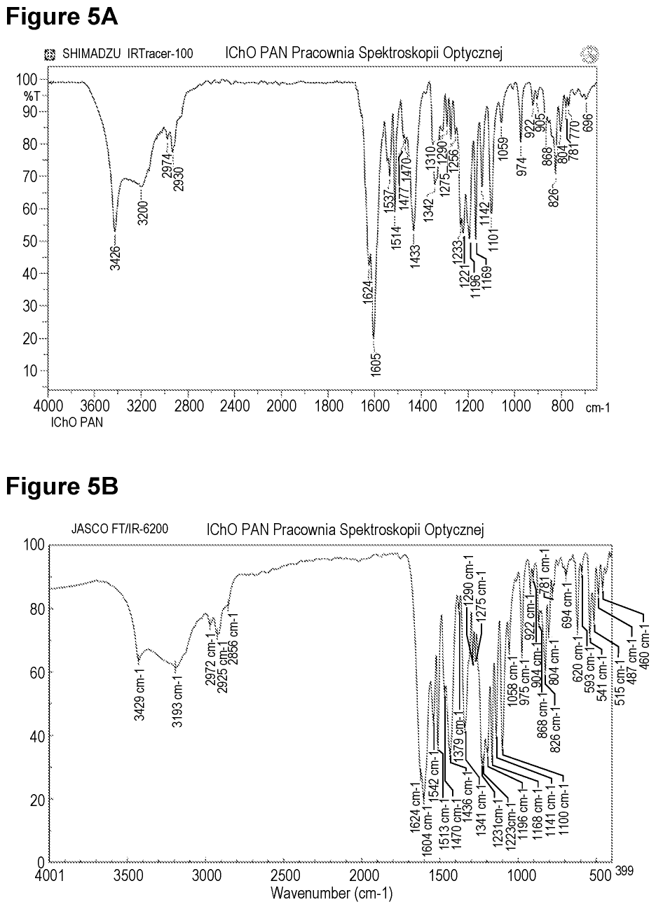

Fourier Transform Infrared Analyses were performed on the following FTIR Spectrophotometers: Shimadzu, type IRTracer-100 (DS, batch XN-54) and JASCO FT/IR-6200 (DS, batch XN-63-10). FIGS. 5A and 5B present results of the FTIR analyses of DS batch XN-54 and XN-87, respectively.

### X-Ray Powder Diffraction (XRPD)

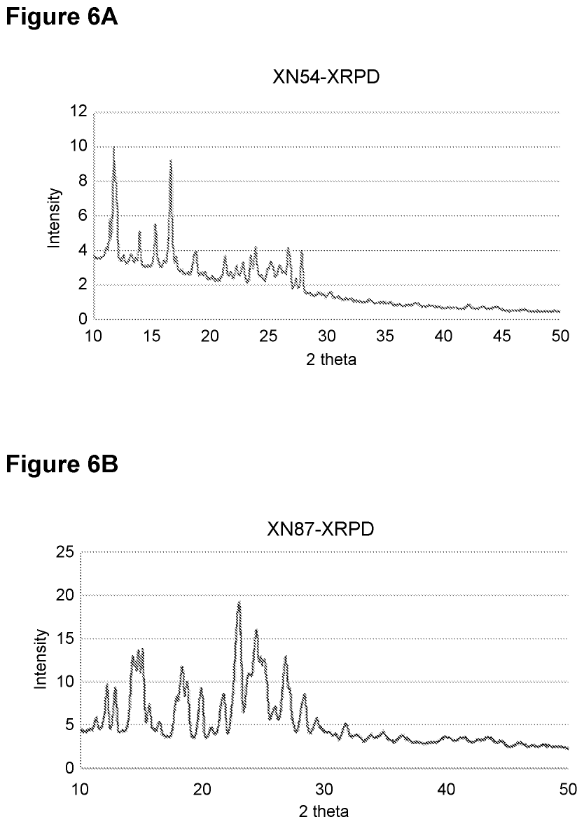

X-Ray powder diffraction analysis indicated crystalline nature of the drug substance and showed two different crystalline forms for two different batches. The diffractogram of XN-54 is shown in FIG. 6A and for XN-87 in FIG. 6B.

### Drug Substance Solubility

Drug substance solubility tests were carried out in 5 media: water, 0.1N hydrochloric acid, acetate buffer pH=4.5, phosphate buffer pH=6.8 and additionally in artificial saliva. The effect of the addition of various surfactants on the solubility of the material was also investigated. The tests were carried out at room temperature, for 2samples of each material in each medium with the use of a magnetic stirrer (excluding preliminary research, where orbital shaker was used). Buffers of pH=4.5 and pH=6.8were prepared according to European Pharmacopeia 10.5, 5.17.1. Artificial saliva was prepared according to the publication: Artificial saliva and its use in biological experiments J. Pytko-Polonczyk1, A. Jakubik, A. Przeklasa-Bierowiec, B. Muszynska, Journal of Physiology and Pharmacology 2017, 68, 6, 807-813. Content of the drug substance in solutions were analyzed by HPLC.

Table 3 presents the HPLC conditions for determination of the content of the drug substance in samples from the solubility test.

TABLE 3
HPLC method parameters for the determination
of dissolved drug substance

| PARAMETER | UNIT | VALUE |
|---|---|---|
| Column | — | Reprosil Gold 120 C18; |
| | | 100 × 4.6 mm; |
| Column temperature | ° C. | 17 |
| Autosampler temperature | ° C. | 20 |
| Flow rate | ml/min | 1.5 |
| Isocratic program | — | A: 75% |
| | | B: 25% |
| Mobile phase A | — | ACN:H₂O:HCOOH - 44:55.8:0.2 |
| Mobile phase B | — | ACN:HCOOH - 99.8:0.2 |
| Detection / wavelength | nm | UV / 370 |
| Injection Volume | μl | 3 / 1* |
| Rinse solution | — | ACN 100% |
| Analysis time | min | 8 |

*3 μl for drug substance solution / 1 μl for granulate solution

### Drug Product Formulation

XN 87 was advanced for formulation development using three formats as shown in Table 4.

TABLE 4
Drug substance formulation

| | | % w/w | | |
|---|---|---|---|---|
| Compound | Function | PIK-12-007 | PIK-12-008 | PIK-12-009 |
| Intragranular phase | | | | |
| XN 87 | Drug Substance | 66.7 | 76.9 | 66.7 |
| Starch 1500 | Filler/binder | 16.7 | — | 20.0 |
| PVP | Filler/binder | — | 2.5 | — |
| Croscarmellose sodium | Superdisintegrant | — | — | 1.0 |
| SDS | Surfactant | 1.3* | 1.2** | 1.3** |
| water | Granulation liquid | q.s. | q.s. | q.s. |
| Extragranular phase | | | | |
| Microcrystalline cellulose | Filler/binder/disintegrant | 11.6 | 15.7 | 7.3 |
| Croscarmellose sodium | Superdisintegrant | 3.0 | 3.0 | 3.0 |
| Magnesium stearate | Lubricant | 0.7 | 0.7 | 0.7 |

*added in powder
**added as a 3% solution

### Methods of Use

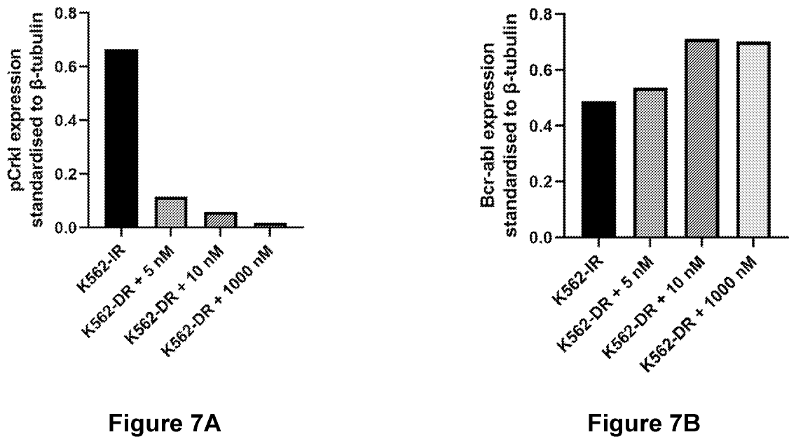

The effect of the drug substance on chronic myelogenous leukemic (CML) cells was tested using a K562 cell line model. Three cell lines were used: K562-IR (imatinib resistant), and K562-IS (imatinib sensitive) were purchased from American Type Culture Collection (ATCC); and K562-DR1000 nM (imatinib & dasatinib resistant) was developed by continuous treatment with TKI and clonal selection. Basal protein expression was confirmed by western blotting of alpha-/beta-tubulin and GAPDH (data not shown). pCrkl levels in K562-DR1000 nM were decreased while BCR-ABL level was similar between dasatinib treated cells (FIG. 7A and 7B, respectively) demonstrating the double-resistance was BCR-ABL independent. Treatment of K562-IS cells with 1000 nM imatinib did not significantly change pCrkl level in comparison with non-treated controls (data not shown).

To measure the effect of XN-54 (Compound 1) on the K562 cell lines, viability experiments were performed using the CellTiter-Glo 2.0 system (Promega) according to the manufacturer's instructions. Briefly, opaque-walled multi-well plates containing the K562 cells in culture medium were incubated with XN-54 at room temperature for the desired times. A volume of CellTiter-Glo reagent equal to the volume of the cell culture medium was added to each well. Luminesence was measured following stabilization of the luminescent signal.

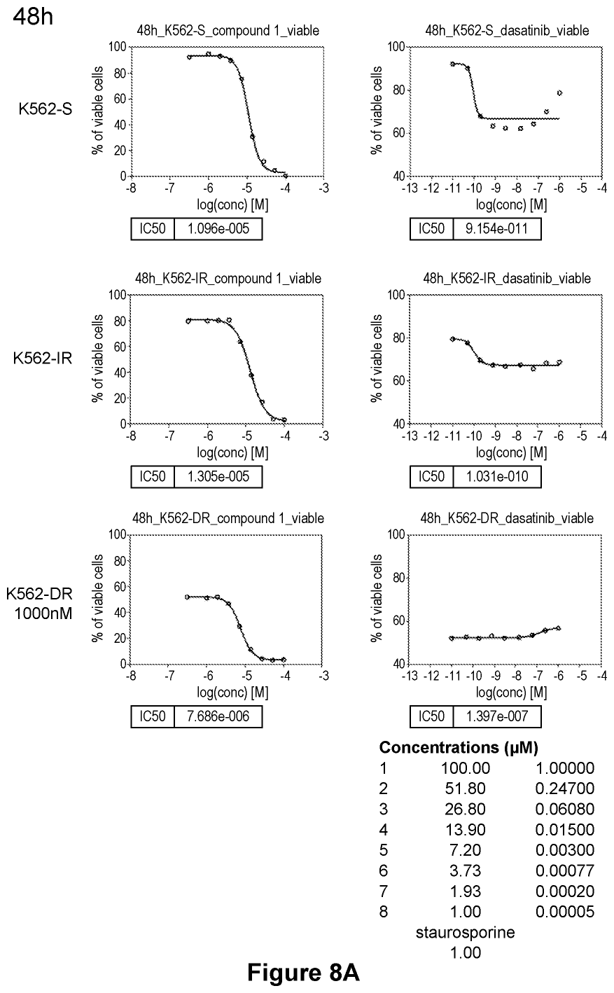

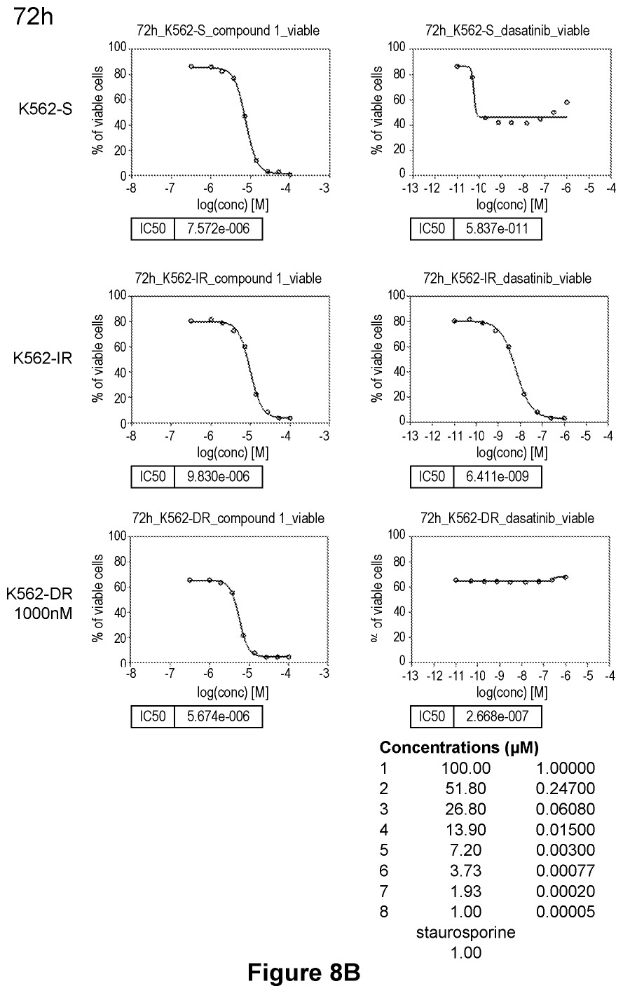

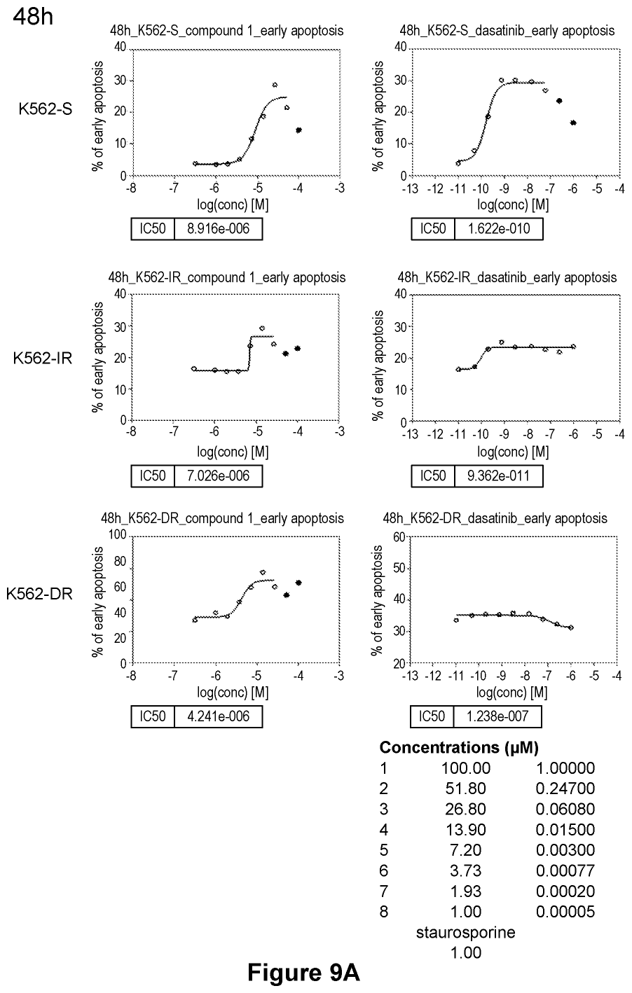

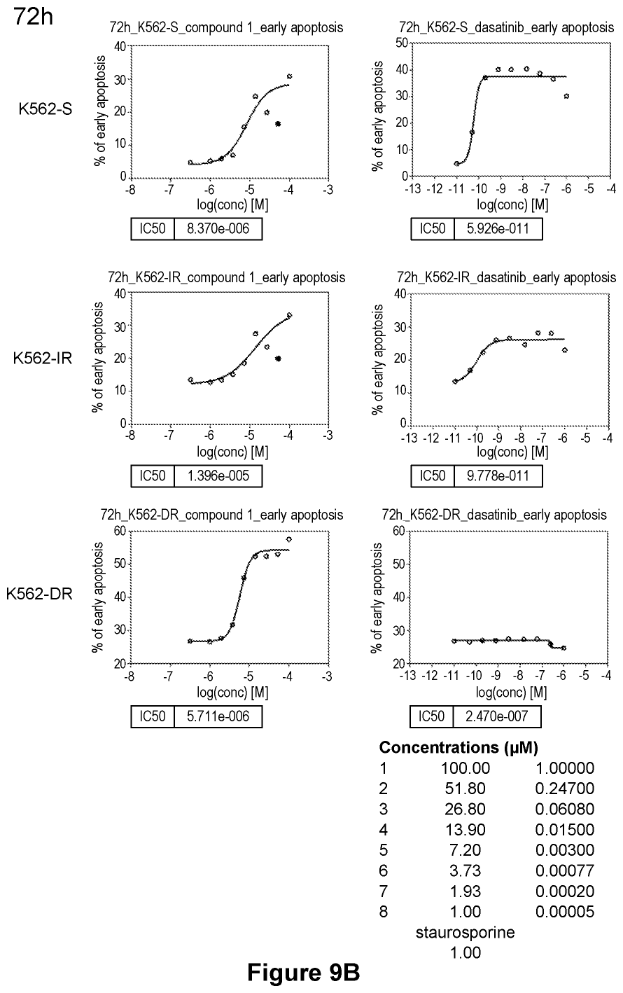

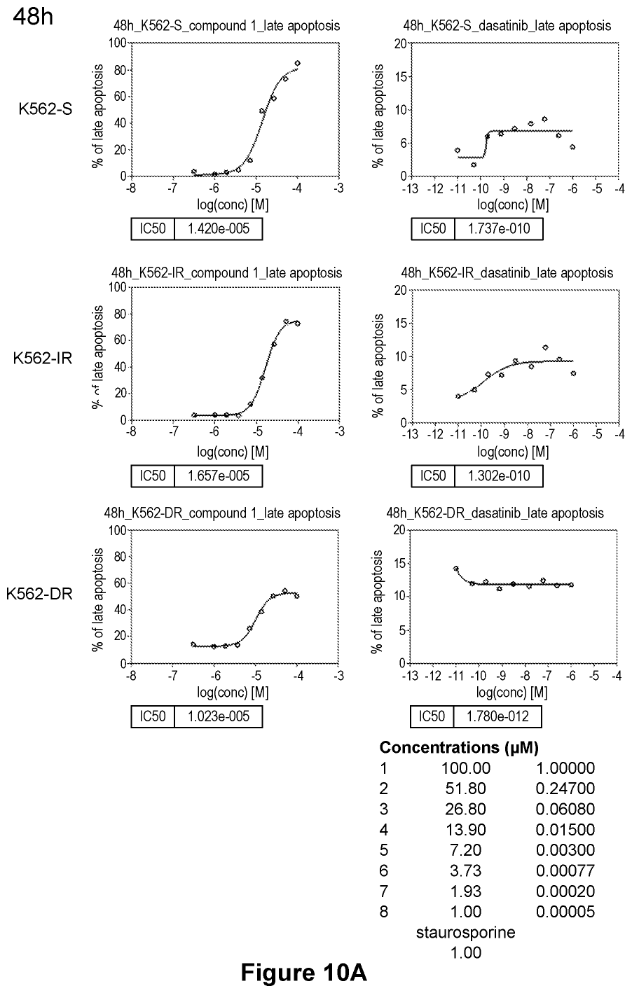

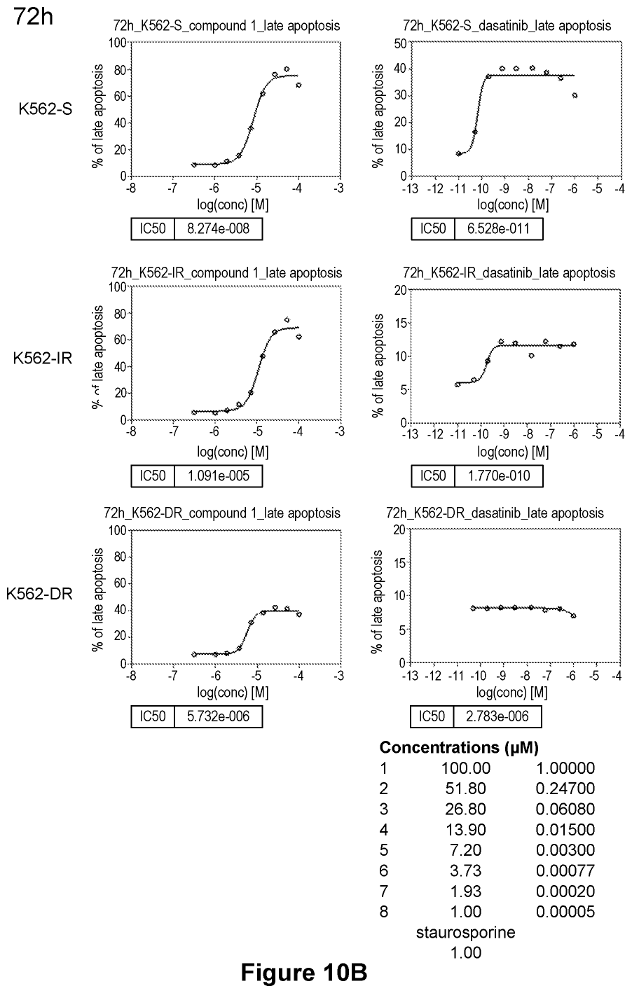

Cell culturing was performed in a 96-well plate format, in 200 μl of medium dedicated for each cell line. Cells were plated at the following densities: 5×10⁵/ml, 2.50×10⁵/ml, 1.25×10⁵/ml, for 24, 48 or 72 hours. Viability, in response to increasing concentrations of XN-54 or dasatinib (control) at both the 48 and 72 hour time points are shown in FIG. 8. As shown in FIG. 8, XN-54 (Compound 1) showed significant activity in both K562 imatinib sensitive and K562 imatinib resistant cells. To determine the mechanism by which XN-54 effected viability, cells were analyzed for apoptosis using both early markers (annexin V) and late markers (annexin V/propidium iodide) (BD Pharmingen cat. 556547). As shown in FIGS. 9 and 10, XN-54 induced apoptosis in all cell lines using both early apoptosis (FIG. 9) and late apoptosis (FIG. 10) markers.

### Combination Therapy

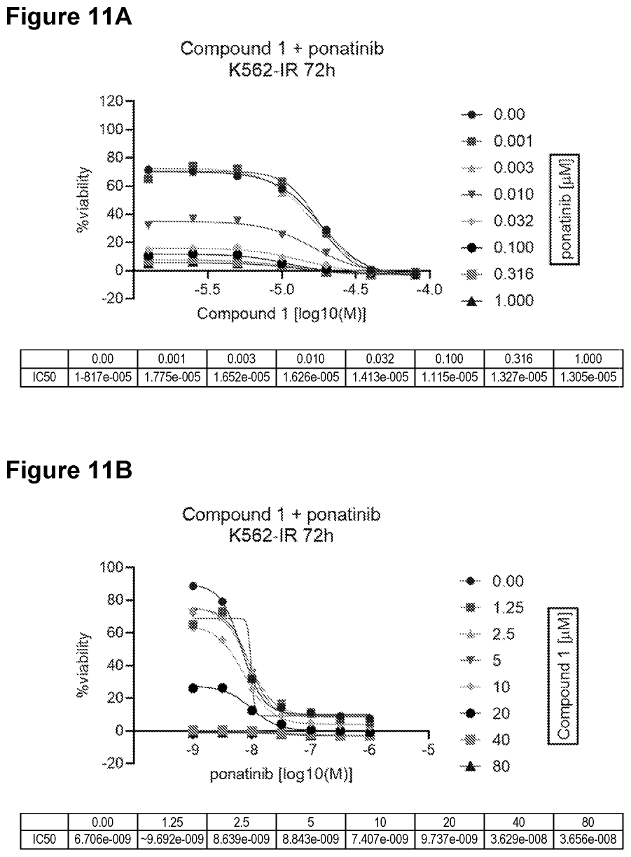

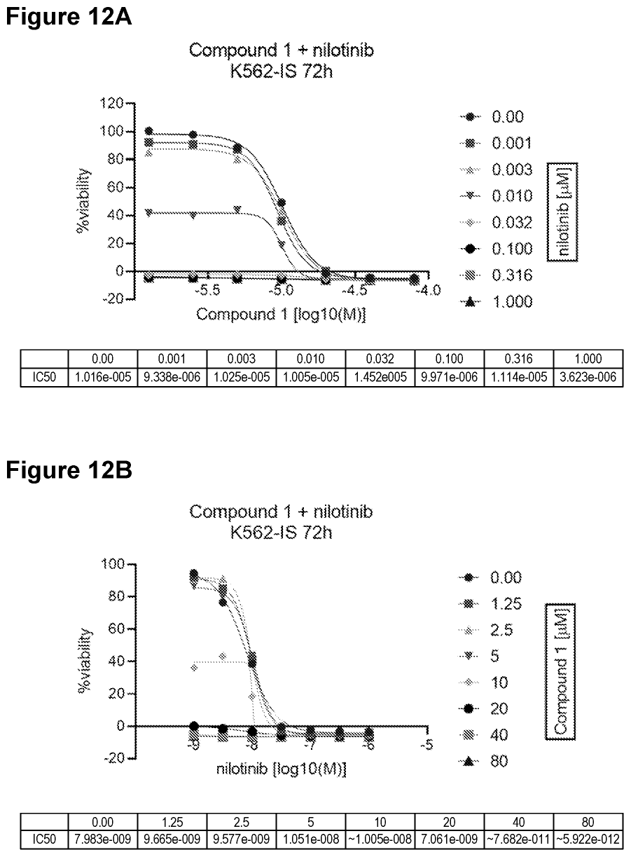

The effect of XN-54 in combination with TKIs was also analyzed on the K562 cells. Percent viability was measured in response to a dose-response combination of XN-54 and ponatinib in K562-IR cells. As shown in FIG. 11, the combination of XN-54 and ponatinib had a significant effect on cell viability based on either log 10(M) XN-54 (FIG. 11A) or ponatinib (FIG. 11B). Similar results were seen in K562-IS cells using XN-54 in combination with nilotinib (FIG. 12).

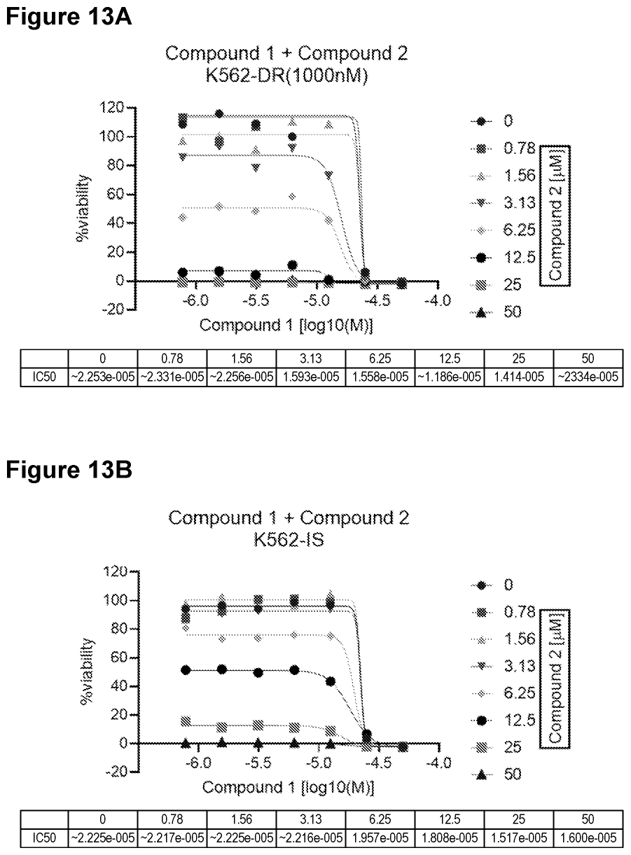

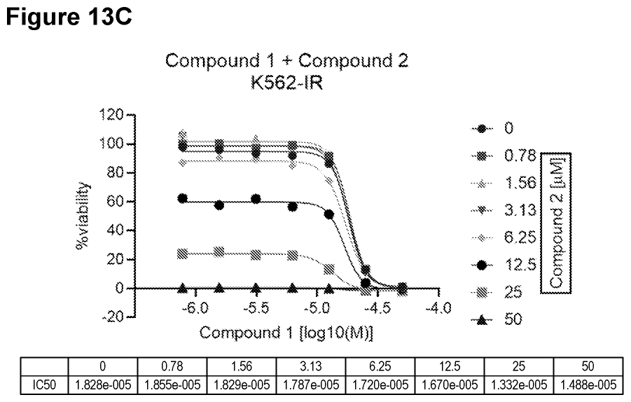

The effect of XN-54 (Compound 1) and cannabidiol (Compound 2) was assayed using the cell viability methods described above. As shown in FIG. 13, the combination of XN-54 and CBD dramatically affected cell viability in K562-DR (FIG. 13A), K562-IS (FIG. 13B), and K562-IR cells (FIG. 13C).

1. A pharmaceutical composition comprising a therapeutically-effective amount of xanthohumol and isoxanthohumol and at least one pharmaceutically acceptable carrier.

2. The composition of claim 1, further comprising 6-prenylnaringenin and 8-prenylnaringenin.

3. The composition of claim 1, wherein the xanthohumol is present in an amount between at least 50-99% w/w, the isoxanthohumol is present in an amount between 1-15% w/w, 6-prenylnaringenin is present in an amount between 0.0-5%, and 8-prenylnaringenin is present in an amount between 0.0-5%.

4-6. (canceled)

7. The composition of claim 1, wherein the pharmaceutically acceptable carrier is at least one of a binder, disintegrant, surfactant, or lubricant.

8. The composition of claim 7, wherein the binder is one or more of starch 1500, polyvinylpyrolidone, and microcrystalline cellulose.

9. (canceled)

10. The composition of claim 7, wherein the disintegrant is croscarmellose sodium.

11. (canceled)

12. The composition of claim 7, wherein the surfactant is sodium dodecyl sulfate.

13. (canceled)

14. The composition of claim 7, wherein the lubricant is magnesium stearate.

15. (canceled)

16. A method of preparing a composition comprising xanthohumol and isoxanthohumol from hops comprising:
suspending the hops plant material inn-heptane to remove non-polar impurities;
evaporating the heptane and filtering the residue;
extracting the residue with ethyl acetate and collecting the xanthohumol-containing fraction in a rotary evaporator;
extracting the xanthohumol with an organic acid solution, containing 0.05M ZnCl₂, 5% NaHCO₃ and brine;
drying the organic layer with anhydrous Na₂SO₄; and
precipitating the xanthohumol from the mixture containing ethyl acetate; and
drying the precipitated xanthohumol and isoxanthohumol.

17. A composition comprising the xanthohumol and isoxanthohumol obtained by the process of claim 16.

18. A method of agonizing Farnesoid X receptor activity in a subject in need thereof, comprising administering a therapeutically effective amount of the composition of claim 1 to the subject.

19-20. (canceled)

21. A method of inhibiting NFκB activity in a subject in need thereof, comprising administering a therapeutically effective amount of the composition of claim 1 to the subject.

22-23. (canceled)

24. A method of increasing expression and/or activation of nuclear factor erythroid 2-related factor 2 (NRF2) in a subject in need thereof, comprising administering a therapeutically effective amount of the composition of claim 1 to the subject.

25-26. (canceled)

27. A method of inducing apoptosis in a cell, comprising contacting the cell with an effective amount of the composition of claim 1.

28. The method of claim 27, wherein the cell is a leukemic cell.

29-30. (canceled)

31. A method of treating chronic myelogenous leukemia (CML) in a subject, comprising administering a therapeutically effective amount of the composition of claim 1 to the subject.

32. The method of claim 31, wherein the CML is resistant to therapy with a tyrosine kinase inhibitor (TKI).

33. (canceled)

34. A method of decreasing the incidence of secondary tyrosine kinase inhibitor (TKI) resistance in a subject comprising administering a therapeutically effective amount of the composition of claim 1 to the subject.

35. A method of treating BCR-ABL-independent resistant cancer in a subject comprising administering a therapeutically effective amount of the composition of claim 1 to the subject.

36. (canceled)

37. The method of claim 18, further comprising administering a TKI to the subject.

38. (canceled)

39. The method of claim 37, wherein the TKI is imatinib, dasatinib, ponatinib, or nilotinib.

40. The method of claim 18, further comprising administering a cannabinoid to the subject.

41. (canceled)
